(19) United States
(12) Patent Application Publication
(10) Pub. No.: US 2011/0311525 A1
(43) Pub. Date: Dec. 22, 2011

(54) DELIVERY OF A CD40 AGONIST TO A TUMOR DRAINING LYMPH NODE OF A SUBJECT

(75) Inventors: Marieke Fernande Herbert-Fransen, Leiden (NL); Cornelis Joseph Maria Melief, Haarlem (NL)

(73) Assignee: ACADEMISCH ZIEKENHUIS LEIDEN H.O.D.N. LUMC, Leiden (NL)

(21) Appl. No.: 13/061,366

(22) PCT Filed: Aug. 31, 2009
(86) PCT No.: PCT/NL2009/050518

§ 371 Date: Feb. 28, 2011

Related U.S. Application Data
(60) Provisional application No. 61/092,792, filed on Aug. 29, 2008.

(30) Foreign Application Priority Data
Aug. 29, 2008 (EP) 08163311.7

(51) Int. Cl. A61K 39/395; A61K 31/7052; A61P 35/00; A61K 38/02

(52) U.S. Cl. 424/133.1; 424/173.1; 514/19.3; 514/44 R

(57) ABSTRACT

The invention relates to the use of a CD40 agonist for treating cancer, a pre-malignant disorder or an infectious disease, wherein a CD40 agonist is locally administered and targeted to a tumor draining lymph node of a subject. Optionally, a CD40 agonist is formulated in a slow-release formulation. Optionally, a CTL-activating peptide is further administered.

### FIELD OF THE INVENTION

The invention relates to the use of a CD40 agonist for treating cancer, a pre-malignant disorder or an infectious disease, wherein a CD40 agonist is locally administered and targeted to (a) tumor draining lymph node(s) of a subject.

### BACKGROUND OF THE INVENTION

Many tumors escape surveillance by our immune system. In cancer patients there is clearly a quantitative and/or qualitative defect in the immune system's specific mechanisms to delete tumor cells. One of these mechanisms is provided by the cytotoxic T cells (CTL) that can recognize and kill cells infected by virus or transformed into cancer cells. It is now known that the T-helper cell does not provide helper signals directly to the CTL (by secretion of IL2), but rather, T-helper cells provide a signal to the Dendritic Cells (DC) that induces only partially characterised cell surface and/or soluble molecules that can activate CTL in the absence of T-helper cells. The signal provided by the T-helper cell to the DC is mediated by CD40L-CD40 interaction. This novel finding has provided a unique opportunity for cancer immunotherapy.

Studies using a CD40 agonist agent have reported that stimulation of the CD40 receptor elicits a cascade of effects associated with anti-tumor activity. For example, stimulation of the CD40 receptor on antigen presenting cells has been shown to enhance their maturation, antigen-presenting function, costimulatory potential and their release of immunoregulatory cytokines (Lee et al., PNAS USA, 1999, 96 (4): 1421-6; Cella et al., J. Exp. Med., 1996, 184 (2): 747-52). The significance of these immune stimulatory and direct anti-tumor effects has been illustrated in animal models in which a CD40 agonist antibody has been shown to prevent tumor growth and reverse tumor tolerance (Diehl et al., Nature Med., 1999, 5 (7): 774-9; Francisco et al., Cancer Res., 2000, 60 (12): 32225-31). Also, systemic administration or intra-tumoral injection of anti-CD40 agonist monoclonal antibody activates DC in tumor-draining lymph nodes. These activated DC trigger a population of inert, so called “poised” tumor-specific T-cells, residing exclusively in tumor-draining lymph nodes, that, as a direct result of the DC mediated activation, now migrate out of the tumor draining lymph node to become systemically circulating tumoricidal effector cells, mediating tumor eradication (van Mierlo et al. 2002; van Mierlo et al., 2004).

The use of a CD40 agonist thus in theory seems very promising. Nevertheless its use in human clinical studies has been associated with toxicity, most importantly cytokine release syndrome, characterised by fever, chills and vascular effects that can be life-threatening and are dose-limiting (Vonderheide et al, Journal of Clinical Oncology, 2007, 25: 876-883). Therefore, there is still a need for using a CD40 agonist for treating cancer wherein said treatment would be less toxic than known treatment with a CD40 agonist.

### DESCRIPTION OF THE INVENTION

The inventors demonstrated that the targeting of a CD40 agonist selectively to (a) tumor draining lymph node(s), which is a form of local administration, has several advantages compared to a classical systemic administration. Although this is a local administration, for example accomplished by subcutaneous or intracutaneous injection of a CD40 agonist in the vicinity of a tumor, it will still induce a systemic anti-tumor response. Without wishing to be bound by any theory, we expect that by selectively delivering a CD40 agonist to a tumor draining lymph node, “poised” T-cell present in a tumor draining lymph node will be activated, turning a local T-cell response into a systemic tumoricidal T-cell response (see above). In addition, as a crucial component of the invention, less toxic effects will be associated with this specific mode of administration, because the dose could be lowered considerably, compared to systemic administration. Indeed very low quantities of a CD40 agonist could still be used to induce a desired anti-tumor effect as defined later herein.

Accordingly, in a first aspect, there is provided the use of an agonist of CD40 for the manufacture of a medicament for treating cancer, a pre-malignant disorder or an infectious disease in a subject wherein the medicament is locally administered and targeted to a tumor draining lymph node of said subject.

### CD40 Agonist

Within the context of this invention, a CD40 agonist is a molecule which specifically binds to the subject's CD40 molecule and increases or enhances or induces one or more CD40 activities by at least about 5% when it comes in contact with a cell, tissue or organism of the subject expressing CD40 in any of the assays as defined below. In some embodiments, an agonist activates one CD40 activity by at least 10%, 20%, 30%, 40%, 50%, 60%, 70%, 80%, or 85% or more. In some embodiments, the activation occurs in the presence of CD40L (CD40 ligand). In some embodiments, an activity of an agonist is measured using a whole blood leukocyte surface molecule upregulation assay. In another embodiment, an activity of an agonist is measured using a dendritic cell assay to measure IL-12 release. In another embodiment, an activity of an agonist is measured by assessing its CTL's activation capacity. CTL activation can be analyzed by assessing cell-surface markers such as CD62L, CD25, CD69 using fluorescently labelled monoclonal antibody and flow cytometry, determining the proliferative capacity to their specific antigen in an in vitro tritium incorporation test, and analyzing the cytokine production with intracellular cytokine staining or ELISA. In another embodiment an activity of an agonist is measured using an in vivo tumor model. In this embodiment, the activity of an agonist is measured by assessing CD8 cytotoxic T-cell activity by tetramer staining of PBMC or lymphoid tissue sections or by intracellular cytokine staining of CD4⁺ and CD8⁺ cells, staining simultaneously for CD4, CD8 and different cytokines, including interferon gamma, IL-4, IL-5 and TNF alpha or by in vivo cytotoxicity assay utilizing intravenously injected spleen target cells stained with different concentration of the colour CFSE and loaded with the specific target peptide or with an irrelevant peptide.

An activity of an agonist of CD40 can be tested by enzyme linked immunosorbent assay (ELISA), Western immunoblotting, or other techniques such as immunochemistry or RNA expression arrays on Dendritic Cells or T-cells.

In a preferred embodiment, a CD40 agonist is an agonist CD40 antibody.

A CD40 agonist of the invention can be made by conventional production and screening techniques. A rat and a hamster anti-mouse CD40 monoclonal antibody (“Mabs”) are each described in Nature 393: 474-77 (1998) and are available commercially (Pharmingen, Inc., CA). The anti-mouse CD40 antibody, designated FGK45, which is used in the experiments described below, is described by Rolink. A. et al., Immunity 5, 319-330 (1996). In a preferred embodiment, to treat a human subject, an anti-human CD40 antibody or human CD40 antibody is used. Such human antibody can be made following techniques well-known in the art, and described by G. Khler and C. Milstein (Nature, 1975: 256: 495-497). As used herein, the term “human antibody” means an antibody in which the variable and constant domain sequences are derived from human sequences. Human CD40 antibodies are described in detail in WO 03/040170. A human antibody provides a substantial advantage in a use of the present invention, as it is expected to minimize the immunogenic and allergic responses that are associated with use of non-human antibodies in a human patient.

An antibody can be raised by immunizing rodents (e.g. mice, rats, hamsters and guinea pigs) with either native CD40 as expressed on cells or purified from human plasma or urine, or recombinant CD40 or its fragments, expressed in a eukaryotic or prokaryotic system. Other animals can be used for immunization, e.g. non-human primates, transgenic mice expressing human immunoglobulins and severe combined immunodeficient (SCID) mice transplanted with human B lymphocytes. Hybridomas can be generated by conventional procedures by fusing B lymphocytes from the immunized animals with myeloma cells (e.g. Sp2/0 and NSO), as described by G. Kohler and C. Milstein, Nature, 1975: 256: 495-497. In addition, an anti-CD40 antibody can be generated by screening of recombinant single-chain Fv or Fab libraries from human B lymphocytes in phage-display systems.

For treating a human subject, an agonistic anti-CD40 antibody would preferably be a chimeric, deimmunised, humanized or human antibodies. Such antibodies can reduce immunogenicity and thus avoid human anti-mouse antibody (HAMA) response. It is preferable that the antibody be IgG4, IgG2, or other genetically mutated IgG or IgM which does not augment antibody-dependent cellular cytotoxicity (S. M. Canfield and S. L. Morrison, J. Exp. Med., 1991: 173: 1483-1491) and complement mediated cytolysis (Y. Xu et al., J. Biol. Chem., 1994: 269: 3468-3474; V. L. Pulito et al., J. Immunol., 1996; 156: 2840-2850).

A chimeric antibody may be produced by recombinant processes well known in the art, and has an animal variable region and a human constant region. A humanized antibody usually has a greater degree of human peptide sequences than do chimeric antibodies.

In a humanized antibody, only the complementarity determining regions (CDRs), which are responsible for antigen binding and specificity are animal derived and have an amino acid sequence corresponding to the animal antibody, and substantially all of the remaining portions of the molecule (except, in some cases, small portions of the framework regions within the variable region) are human derived and correspond in amino acid sequence to a human antibody (see L. Riechmann et al., Nature, 1988; 332:323-327; G. Winter, United States Patent No. C. Queen et al., U.S. Pat. No. 5,530,101).

A deimmunised antibody is an antibody in which the T and B cell epitopes have been eliminated, as described in International Patent Application PCT/GB98/01473. They have reduced immunogenicity when applied in vivo.

A human antibody can be made by several different ways, including by use of human immunoglobulin expression libraries (Stratagene Corp., La Jolla, Calif.) to produce fragments of human antibodies VH, VL, Fv, Fd, Fab, or (Fab′)2, and using these fragments to construct whole human antibodies using techniques similar to those for producing chimeric antibodies. Alternatively, these fragments may be used on their own as agonist. Human antibodies can also be produced in transgenic mice with a human immunoglobulin genome. Such mice are available from Abgenix. Inc., Fremont, Calif., and Medarex, Inc., Annandale, N.J.

One can also create single peptide chain binding molecule in which the heavy and light chain Fv regions are connected. Single chain antibodies (“ScFv”) and the method of their construction are described in U.S. Pat. No. 4,946,778. Alternatively, Fab can be constructed and expressed by similar means (M. J. Evans et al., J. Immunol. Meth., 1995; 184:123-138). All of the wholly and partially human antibodies are less immunogenic than wholly murine MAbs, and the fragments and single chain antibodies are also less immunogenic. All these types of antibodies are therefore less likely to evoke an immune or allergic response. Consequently, they are better suited for in vivo administration in a human subject than wholly animal antibodies, especially when repeated or long-term administration is necessary. In addition, the smaller size of the antibody fragment may help improve tissue bioavailability, which may be critical for better dose accumulation in acute disease indications, such as tumor treatment.

Preferred human anti-CD40 antibody have been extensively described in WO 2005/063289.

Based on the molecular structures of the variable regions of an anti-CD40 antibody, one could use molecular modeling and rational molecular design to generate and screen molecules which mimic the molecular structures of the binding region of the antibodies and activate CTLs. These small molecules can be peptides, peptidomimetics, oligonucleotides, or other organic compounds. The mimicking molecules can be used for treatment of cancers. Alternatively, one could use large-scale screening procedures commonly used in the field to isolate suitable molecules from libraries of compounds. In one embodiment, several CD40 agonists are used simultaneously or sequentially.

### Administration

The invention resides in the way a CD40 agonist is administered to a subject, preferably a human subject. A CD40 agonist is preferably locally administered and targeted to a tumor draining lymph node of a subject. What matters is that a local administration of a CD40 agonist is carried out. In other words, the invention is not directed to a systemic administration of a CD40 agonist. Preferably, the invention defines a specific way of locally administering a CD40 agonist to a subject. The local administration of a CD40 agonist is preferably targeted to a tumor draining lymph node of a subject. In a more preferred embodiment, the local administration targeted to a tumor draining lymph node is realized by administering a CD40 agonist in the vicinity of or into a tumor-draining lymph node. In this context, “in the vicinity” preferably means about a few cm or a few cm or less of a tumor-draining lymph node. In this context, “in the vicinity” preferably means a few cm or less removed from the site of a tumor-draining lymph node. A CD40 agonist is not per se directly administered into a tumor-draining lymph node. However, the administration of a CD40 agonist is such that the administered CD40 agonist will preferably be selectively delivered into a tumor-draining lymph node. A CD40 agonist is preferably indirectly administered into or selectively administered into or targeted to a tumor-draining lymph node: it means it is not directly administered into a tumor-draining lymph node, but the way it is administered will preferably result in the fact that at least 30% of the initially administered CD40 agonist will reach a tumor-draining lymph node. Preferably, at least 40%, 50%, 60%, 70%, 80%, 90%, 95%, 98%, 99% or 100%. The presence of a CD40 agonist in a tumor-draining lymph node is preferably assessed by immunostaining on a biopsy or bio-imaging of a specific CD40 agonist antibody (preferably the FGK 45 as identified in the example) labelled with a fluorescent group in the range of 680-700 nm (for instance an ALEXA-fluor group), said antibody being injected into a subject, where during surgery the fluorescently labeled antibodies can be detected with a camera (camera guided surgery).

In a preferred embodiment, a CD40 agonist is not administered intratumorally. Intratumoral administration is not always preferred since each tumor is different (i.e. vascularisation, tissue distribution, osmostic pressure . . . ) and therefore an intratumoral administration of a compound can not be standardized and the therapeutic effects may be unpredictable.

In a preferred embodiment, an agonist of CD40 is locally administered and targeted to a tumor draining lymph node of a subject via subcutaneous or intracutaneous injection. More preferably, a subcutaneous or intracutaneous injection is carried out directly to a tumor draining lymph node of a subject.

In another preferred embodiment, the location of injection is located in the area between a tumor and the nearest tumor-draining lymph node, or in a tumor-draining lymph node directly.

In another preferred embodiment, a CD40 agonist is administered into a lymphatic vessel. More preferred lymphatice vessel is the one at the dorsum of the foot. In this more preferred embodiment, an agonist of CD40 is locally administered and targeted to a tumor draining lymph node of a subject by a para-aortal injection in a lymph node of said subject using similar techniques as used when performing a lymphangiography (Guermazi et al., Radiograph. 2003: 23: 1541-1560 and Pollen et al., Cancer Suppl. 2003: 98: 2028-2038). By the methodology of lymphangiography, administration of a drug, in this case a CD40 agonist, is performed at the dorsum of the foot after which it travels selectively along the lymphatic channels into the lymph nodes into which these lymphatic vessels drain. In the case of administration at the dorsum of the foot (Follen et al., Cancer Suppl. 2003: 98: 2028-2038), it will selectively target to the lymph nodes of the pelvis and after that the para-aortal nodes. This is an advantageous way of administration for the treatment of gynecological tumor as later defined herein.

The invention therefore encompasses an injection into the dorsum of a foot, an injection into a lymph node of the pelvis (directly or indirectly as a result of the injection into the dorsum of a foot), a para-aortal injection (directly or indirectly via an injection into the dorsum of a foot or via an injection into a lymph node of the pelvis).

Within the context of the invention, subcutaneous injection preferably means subcutaneous injection in the vicinity of a tumor, said tumor preferably having a subcutaneous or intracutaneous localization. Within the context of the invention, intracutaneous injection preferably means intracutaneous injection in the vicinity of a tumor, said tumor preferably having a subcutaneous or intracutaneous localization. In a further preferred embodiment, an agonist of CD40 is locally administered and targeted to a tumor draining lymph node through a lymph vein injection. This is a technique known to the skilled person, such as a person skilled in the art of lymphangiography.

It is further encompassed by the invention to locally administer and target a CD40 agonist at one or more tumor draining lymph node(s) sequentially or simultaneously, preferably subcutaneously. It is also encompassed by the invention to locally administer and target a CD40 agonist at one tumor draining lymph node, preferably subcutaneously, intracutaneously and/or through several direct lymph vein injections as in lymphangiography.

The local administration and the targeting to a tumor draining lymph node have several advantages. First of all, it will deliver a CD40 agonist almost directly to DCs which are present in a tumor draining lymph node. Such activated DCs will in turn activate CTL as known to the skilled person. Second, since this is a local administration, we expect toxicity will be reduced. This has been specifically demonstrated in the examples. Third, this local administration allows the use of a lower dose of a CD40 agonist as demonstrated herein and as extensively explained herein. Fourth, surprisingly, although this is a local administration, a systemic activation of the immune system has been demonstrated in the examples.

The use of an agonist of CD40 as identified herein preferably leads to a therapeutic effect. A therapeutic effect may be an anti-tumor and/or an anti-infectious effect. An anti-tumor effect is preferably identified as:
an activation or an induction of the systemic immune system: detectable and/or an increase in tumor specific activated CD4⁺ or CD8⁺ T-cells in peripheral blood or an increase thereof or of the cytokines produced by these T-cells after at least one week of treatment and/or
an inhibition of proliferation of tumor cells and/or
an induction or increased induction of tumor cells death and/or
an inhibition or prevention or delay of the increase of a tumor weight or growth and/or
a prolongation of patient survival of at least one month, several months or more (compared to those not treated or treated with an isotype control).

A significant increase of tumor-specific activated CD4⁺ or CD8⁺ cells in peripheral blood after at least one week of treatment may be of at least 5%, 10%, 20%, 30% or more. An inhibition of the proliferation of tumor cells may be at least 20%, 30%, 40%, 50%, 55%, 60%, 65%, 70% or 75%, or more. An induction of tumor cell death may be at least 1%, 5%, 10%, 15%, 20%, 25%, or more. Tumor growth may be inhibited at least 5%, 10%, 20%, 30%, 40%, 50%, 55%, 60%, 65%, 70% or 75%, or more. In certain embodiments, tumor weight increase may be inhibited at least 20%, 30%, 40%, 50%, 55%, 60%, 65%, 70% or 75%, or more. In certain embodiments, tumor growth may be delayed at least one week, one month, two months or more.

The use of an agonist of CD40 as identified herein preferably leads to an anti-infectious effect. An anti-infectious effect is preferably identified as:
an activation or an induction of the systemic immune system: detectable and/or an increase in specific activated CD4⁺ or CD8⁺ T-cells in peripheral blood that are specifically directed against an infectious agent or against an infected cell (i.e. called herein infection-specific activated CD4⁺ or CD8⁺ cells) or an increase thereof or of the cytokines produced by these T-cells after at least one week of treatment and/or
an inhibition of proliferation of infected cells or of an infectious agent and/or
an induction or increased induction of the death of infected cells or of an infectious agent and/or
an inhibition or prevention or delay of the increase of the number of infected cells or of an infectious agent and/or
a prolongation of patient survival of at least one month, several months or more (compared to those not treated or treated with an isotype control).

A significant increase of infection-specific activated CD4⁺ or CD8⁺ cells in peripheral blood after at least one week of treatment may be of at least 5%, 10%, 20%, 30% or more. An inhibition of the proliferation of infected cells (or infectious agent) may be at least 20%, 30%, 40%, 50%, 55%, 60%, 65%, 70% or 75%, or more. An induction of the death of infected cells (or infectious agent) may be at least 1%, 5%, 10%, 15%, 20%, 25%, or more. The increase of the number of infected cells may be inhibited at least 5%, 10%, 20%, 30%, 40%, 50%, 55%, 60%, 65%, 70% or 75%, or more. In each embodiment wherein the effect of an agonist of CD40 is quantified, the assay may be carried out by comparison to a subject not treated or to the same subject before treatment or compared to a subject treated with an immunoglobulin isotype control antibody. In some embodiments, a tumor is CD40 positive. In some embodiments, a tumor is CD40 negative. A tumor can be a solid tumor or a non-solid tumor such as lymphoma. Some types of tumors or infection that can be treated using the present invention are extensively identified later herein.

The dosage for an agonist of the invention can be readily determined by extrapolation from the in vitro tests and assays described below, or from animal experiments or from human clinical trials. We demonstrated that the local administration of a dose of a given agonist of CD40 targeted to a tumor draining lymph node could induce the same anti-tumor effect as using a systemic administration of a higher dose of the same agonist. Therefore, the invention allows the use of a lower dose of a CD40 agonist. “Lower” preferably means approximately 2-20% of the dose (quantity) of an agonist of CD40 as administered systemically. Lower may also mean approximately 30 to 60%, 40 to 70%, or 50% to 80% of an agonist. Lower may also mean approximately 20 to 40%, 15 to 30%, or 10% to 20% of an agonist. “Lower” preferably means 2-20% of the dose (quantity) of an agonist of CD40 as administered systemically. Lower may also mean 30 to 60%, 40 to 70%, or 50% to 80% of an agonist. Lower may also mean 20 to 40%, 15 to 30%, or 10% to 20% of an agonist. In a preferred embodiment, a dose of at least 20 μg CD40 agonist is locally administered in a single dose and targeted to a tumor draining lymph node, preferably at least 30 μg, at least 40 μg, at least 50 μg, at least 60 μg, at least 70 μg, at least 80 μg, at least 90 μg, at least 100 μg. In a further preferred embodiment, a single dose of not more than 100 μg is locally administered and targeted to a tumor draining lymph node, not more than 90 μg, not more than 80 μg, not more than 70 μg, not more than 60 μg, not more than 50 μg, not more than 40 μg, not more than 30 μg, not more than 20 μg. Very good results were obtained with a single dose of 30 μg of a CD40 agonist.

A subject that can be treated with a CD40 agonist includes, but is not limited to a subject that has been diagnosed as having a cancer, a pre-malignant disorder or an infectious disease. Examples of cancer include brain cancer, lung cancer, bone cancer, pancreatic cancer, skin cancer, cancer of the head and neck, cutaneous or intraocular melanoma, uterine cancer, ovarian cancer, rectal cancer, cancer of the anal region, stomach cancer, gastric cancer, colorectal cancer, colon cancer, gynecologic tumors (e.g. uterine sarcomas, carcinoma of the fallopian tubes, carcinoma of the endometrium, carcinoma of the cervix, carcinoma of the vagina or carcinoma of the vulva, HPV derived cancer), cancer of the esophagus, cancer of the small intestine, cancer of the endocrine system (e.g., cancer of the thyroid, parathyroid or adrenal glands), sarcomas of soft tissues, leukemia, myeloma, multiple myeloma, cancer of the urethra, cancer of the penis, prostate cancer, chronic or acute leukemia, solid tumors of childhood, Hodgkin's disease, lymphocytic lymphomas, non-Hodgkin lymphoma, cancer of the bladder, liver cancer, renal cancer, cancer of the kidney or ureter (e.g., renal cell carcinoma, carcinoma of the renal pelvis), or neoplasms of the central nervous system (e.g., primary CNS lymphoma, spinal axis tumors, brain stemgliomas or pituitary adenomas), glioma or fibrosarcoma. Examples of an infectious disease include infections which may lead to a cancer such an HPV, HCV, HBV, HTLV I, Herpesvirus type 8 (Kaposi sarcoma agent), EBV or HIV infection.

As used herein, the term “subject” preferably refers to a human or a non-human mammal that expresses a cross-reacting CD40 (e.g., a primate, cynomolgus or rhesus monkey). Preferably a subject being treated is a human.

In a preferred embodiment, one single administration of an agonist of CD40 is locally administered and targeted to a tumor draining lymph node. In the prior art, usually several sequential, systemic administration of a CD40 agonist are used to obtain a given effect (see for example WO 2005/063289). This is quite inconvenient and complicated for the subject. In addition toxicity is usually quite high. Surprisingly, the inventors found that a single administration of a CD40 agonist locally administered and targeted to a tumor draining lymph node was active enough to induce a systemic activation of the immune system to get a specific anti-tumoral or anti-infectious response as demonstrated in the examples. In addition, less to no toxicity effects accompanied this administration of a CD40 agonist.

In a further preferred embodiment, a CD40 agonist is formulated in a so-called slow release formulation or slow release vehicle. Such formulations are also named formulation with a delayed or controlled release. A controlled release formulation is a formulation that will release at least 20%, at least 25%, at least 30%, at least 35%, at least 40%, at least 45%, at least 50%, at least 55%, at least 60%, at least 65%, at least 70%, at least 75%, at least 80% of its active ingredient in a controlled fashion, i.e. a CD40 agonist within a day, a week, two weeks, three weeks, a month, or longer. The release rate can be adjusted by the ratio between dextran-molecules and cross-linker, and the content of water within the formulation and can be adapted depending on the required period of exposure to the therapeutic compound. A preferred cross-linker is methacrylate. This type of formulation has several advantages: first it is expected that there would be no need to repeat the administration of a CD40 agonist, since once administered in such type of formulation, a CD40 agonist will be delivered over extended period of time when released from this formulation. Such extended period of time may vary between one day, one week, one month to several months depending from the type of slow release formulation used. Second, toxicity is expected to be further reduced since very low local quantities of CD40 agonists are expected to be detectable. We expect that such low local quantities may be less than tenfold of the dose required for systemic treatment with a same antibody. Such “low” quantities are not expected to induce any toxicity but are expected to be functional in inducing an anti-tumor or anti-infectious effect as defined herein. The invention is not limited to a specific type of slow release formulation. Several types of slow release formulations are already known such as mineral oil (e.g. Montanide ISA 51) or Poly-lactic-co-glycolic acid (PLGA) or polymer based formulations. An example of a polymer-based formulation is a gel composition comprising charged polymers as described in WO 2005/110377 or a composition comprising a dextran hydrogel as described in WO 02/17884 or WO 2005/051414 or U.S. Pat. No. 3,710,795. In a preferred embodiment, a CD 40 agonist is formulated with a dextran hydrogel comprising 30%, 40%, 50%, 60% water content. More preferably, the water content is ranged between 45% and 55%, more preferably is approximately 50% or is 50%. Preferably 2, 3, 4, 5, 6, 7, 8, 9, 10 μg of a CD40 agonist is formulated in such dextran hydrogel. A dextran hydrogel having a water content of 50% with 5 μg of a CD40 agonist has been found to be attractive in the experimental part: it seems to exhibit the slowest possible formulation, no CD40 agonist is detectable in the serum whereas an effect on a T cell response could be detected (see experimental part).

In another further preferred embodiment, a CD40 agonist is linked or fused to or associated with or mixed with a compound that will be specifically recognized by DC. In this way the targeting of a CD40 agonist to DC within a lymph node is expected to be further improved. An example of such a compound is a ligand for the DC-SIGN C-type lectin on DC, which will bind DC-SIGN present at the surface of DC. Another example is a ligand for the DEC-205 molecule on DC. (Bozzacco, L., Trumpfheller, C., Siegal, F. P., Mehandru, S., Markowitz, M., Carrington, M., Nussenzweig, M. C., Piperno, A. G., and Steinman, R. M. (2007). DEC-205 receptor on dendritic cells mediates presentation of HIV gag protein to CD8+ T cells in a spectrum of human MHC I haplotypes. Proc. Natl. Acad. Sci. U.S. A 104, 1289-1294.)

### Other Molecule/Treatment

In an embodiment, a CD40 agonist is used (simultaneously or sequentially) with another molecule and/or another treatment. Examples of another treatment include another classical cancer treatment such as chemotherapy, radiotherapy. Examples of another molecule include a DNA replication inhibitor such as cisplatin and/or a peptide, preferably a CTL-activating peptide and/or a T helper activating peptide and/or another compound.

In a preferred embodiment a CD40 agonist is used in combination with another compound or molecule, which is able to stimulate the immune system, i.e. an immune stimulatory compound, hereafter named second stimulating compound. An activation or an induction of the immune system, preferably the systemic immune system by said second stimulating compound has been earlier defined herein. Preferred second compound is an antibody. Preferred antibodies include a CTLA4-blocking antibody, an anti-OX40 activating antibody and an anti-41BB activating antibody. ACD40 agonist and a second stimulating compound may be administered simultaneously or sequentially. More preferably, a CD40 agonist and a second stimulating compound are formulated in one single composition, even more preferably in a slow release formulation as defined earlier herein. The use of these two or more compounds allows a synergistic activation of T cells as demonstrated in the examples. Preferred CTLA4-blocking antibodies that can be used in human are described in Camacho et al, J. Clin. Oncol. (2009), 27:1075-1081.

A CTL-activating peptide used in combination with a CD40 antibody has been extensively described in WO 99/61065. A CTL-activating peptide or a T helper activating peptide is preferably a tumor-derived or virus-derived peptide. A CTL-activating peptide or a T helper activating peptide is not supposed to be limited to any length. However, it is preferred that such peptide has a length which is comprised within 19 and 45 amino acids. Said amino acid sequence being preferably entirely or partly derived from a protein expressed by a tumor cell. The length of the contiguous amino acid sequence derived from a protein comprised within the peptide, preferably is comprised between 19-45, 22-45, 22-40, 22-35, 24-43, 26-41, 28-39, 30-40, 30-37, 30-35, 32-35 33-35, 31-34 amino acids. In another preferred embodiment, a peptide comprises 22, 23, 24, 25, 26, 27, 28, 29, 30, 31, 32, 33, 34, 35, 36, 37, 38, 39, 40, 41, 42, 43, 44, or 45 or more than 45 contiguous amino acid residues of a protein. Preferred CTL-activating peptides or T helper activating peptides are derived from a HPV protein when the cancer is a HPV-related cancer and/or the infection is a HPV infection.

In another preferred embodiment, a CTL-activating peptide or a T helper activating peptide consists of any of the contiguous amino acid sequences of a length of 9-45 amino acids derived from the amino acid sequence of a tumor-associated protein such as HPV E2, E6 and E7, p53, PRAME, NY-ESO-1, or any other tumor—associated or tumor-specific protein or any infectious-associated or infectious-specific protein. The amino acid sequence of the HPV serotype 16 E2, E6 and E7 proteins are depicted in SEQ ID No. 1, 2 and 3 respectively. The amino acid sequence of the HPV serotype 18 E2, E6 and E7 proteins are depicted in SEQ ID No. 4, 5 and 6 respectively. The amino acid sequence of human p53 is depicted in SEQ ID No. 7.

Preferred CTL-activating or T helper activating peptides are derived from HPV E2, E6 or E7. In the experimental part, two peptides derived from different tumor associated proteins are used as examples of suitable peptides to be used in the context of the invention: one is identified as long synthetic CEA peptide and is derived from Carcinoembryonic Antigen (CEA), which is overexpressed in multiple different epithelial tumor types, and the second peptide, long synthetic HPV peptide, is derived from the HPV E7 protein. More preferred CTL-activating or T helper activating peptides are derived from HPV E2, E6 or E7 are disclosed in WO 02/070006. Preferably, a CTL-activating or a T helper activating peptide comprising or consisting of a contiguous amino acid sequence selected from the full length amino acid sequences of the HPV E2, E6 or E7 proteins is from a high risk HPV serotype, such as serotypes 16, 18, 31, 33 or 45, more preferably from the amino acid sequences of the HPV E6 and E7 serotypes 16, 18, 31 or 33, most preferably from serotypes 16 or 18, of which 16 is most preferred.

Preferred CTL-activating or T helper activating peptides derived from E2 consist of, or comprise amino acids 46-75 of an HPV E2 protein, amino acids 51-70 of an HPV E2 protein, amino acids 61-76 of an HPV E2 protein, amino acids 151-195 of an HPV E2 protein, amino acids 316-330 of an HPV E2 protein, amino acids 311-325 of an HPV E2 protein, amino acids 326-355 of an HPV E2 protein, amino acids 346-355 of an HPV E2 protein, amino acids 351-365 of an HPV E2 protein.

Preferred CTL-activating or T helper activating peptides derived from E6 consist of, or comprise amino acids 1-32 of an HPV E6 protein, amino acids 11-32 of an HPV E6 protein, amino acids 13-22 of an HPV E6 protein, amino acids 19-50 of an HPV E6 protein, amino acids 29-38 of an HPV E6 protein, amino acids 37-68 of an HPV E6 protein, amino acids 41-65 of an HPV E6 protein, amino acids 52-61 of an HPV E6 protein, amino acids 51-72 of an HPV6 protein, amino acids 55-80 of an HPV E6 protein, amino acids 55-86 of an HPV E6 protein, amino acids 61-82 of an HPV E6 protein, amino acids 71-92 of an HPV E6 protein, amino acids 71-95 of an HPV E6 protein, amino acids 73-105 of an HPV E6 protein, amino acids 85-109 of an HPV E6 protein, amino acids 91-112 of an HPV E6 protein, amino acids 91-122 of an HPV E6 protein, amino acids 101-122 of an HPV E6 protein, amino acids 109-140 of an HPV E6 protein, amino acids 121-142 of an HPV E6 protein, amino acids 129-138 of an HPV E6 protein, amino acids 127-140 of an HPV protein, amino acids 127-158 of an HPV E6 protein, amino acids 129-138 of an HPV E6 protein, amino acids 137-146 of an HPV E6 protein, amino acids 149-158 of an HPV E6 protein,

Preferred CTL-activating or T helper activating peptides derived from E7 consist of, or comprise amino acids 1-32 of an HPV E7 protein, amino acids 1-35 of an HPV E7 protein amino acids 11-19 of an HPV E7 protein, amino acids 21-42 of an HPV E7 protein, amino acids 22-56 of an HPV E7 protein amino acids 35-77 of an HPV E7 protein, amino acids 35-50 of an HPV E7 protein, amino acids 50-62 of an HPV E7 protein, amino acids 43-77 of an HPV E7 protein, amino acids 51-72 of an HPV E7 protein, amino acids 64-98 of an HPV E7 protein amino acids 76-86 of an HPV E7 protein.

Another preferred CTL-activating or a T helper activating peptide is derived from a p53 protein, preferably human p53. Preferred CTL-activating or T helper activating peptides derived from p53 consist of, or comprise amino acids 86-115 of a p53 protein, amino acids 102-131 of a p53 protein, amino acids 101-110 of a p53 protein, amino acids 112-120 of a p53 protein, amino acids 113-120 of a p53 protein, amino acids 113-122 of a p53 protein, amino acids 117-126 of a p53 protein, amino acids 142-171 of a p53 protein, amino acids 149-157 of a p53 protein, amino acids 154-163 of a p53 protein, amino acids 154-164 of a p53 protein, amino acids 156-163 of a p53 protein, amino acids 156-164 of a p53 protein, amino acids 157-186 of a p53 protein, amino acids 172-181 of a p53 protein, amino acids 190-219 of a p53 protein, amino acids 196-205 of a p53 protein, amino acids 205-214 of a p53 protein, amino acids 224-248 of a p53 protein, amino acids 225-254 of a p53 protein, amino acids 241-270 of a p53 protein, amino acids 257-286 of a p53 protein, amino acids 229-236 of a p53 protein, amino acids 264-272 of a p53 protein, amino acids 264-272 of a p53 protein, amino acids 273-302 of a p53 protein, amino acids 283-291 of a p53 protein, amino acids 305-334 of a p53 protein, amino acids 311-319 of a p53 protein, amino acids 311-320 of a p53 protein, amino acids 312-319 of a p53 protein, amino acids 322-330 of a p53 protein, amino acids 340-348 of a p53 protein, amino acids 353-382 of a p53 protein, amino acids 360-370 of a p53 protein, amino acids 363-370 of a p53 protein, amino acids 363-372 of a p53 protein, amino acids 369-393 of a p53 protein, amino acids 373-381 of a p53 protein, amino acids 374-382 of a p53 protein, amino acids 376-386 of a p53 protein.

The invention further encompasses a CTL-activating or a T helper activating peptide whose amino acid sequence has at least 50%, 55%, 60%, 65%, 70%, 75%, 80%, 85%, 90%, 95%, 97%, 99% identity with one of the sequences identified herein and wherein this peptide is not the HPV E2, E6, E7 or p53 protein. Preferably a peptide is defined by its identity to one of the identified sequences and has a length as earlier identified herein. Identity is calculated by defining the number of identical amino acids between the two sequences after having aligned both sequences to ensure highest number of identical amino acids will be obtained.

A peptide of such length used in the invention may be easily synthesized. The art currently knows many ways of generating a peptide. The invention is not limited to any form of generated peptide as long as the generated peptide comprises, consists or overlaps with any of the given sequences and had the required activity as earlier defined herein. A peptide may be present as a single peptide or incorporated into a fusion protein. A peptide may further be modified by deletion or substitution of one or more amino acids, by extension at the N- and/or C-terminus with additional amino acids or functional groups, which may improve bio-availability, targeting to T-cells, or comprise or release immune modulating substances that provide adjuvant or (co)stimulatory functions. The optional additional amino acids at the N- and/or C-terminus are preferably not present in the corresponding positions in the amino acid sequence of the protein it derives from. Alternatively, tumor cells may be isolated from a subject to be treated and CTL-activating peptides may be identified from these tumor cells and subsequently formulated as short or long synthetic peptides.

In a further preferred embodiment, a CD40 agonist and optionally a CTL-activating peptide and/or a T-helper cell-activating peptide are formulated as a composition. Preferably, a composition is a pharmaceutical composition. Such a pharmaceutical composition preferably further comprises a pharmaceutical excipient and/or an immune modulator. Any known inert pharmaceutically acceptable carrier and/or excipient may be added to the composition. Formulation of medicaments, and the use of pharmaceutically acceptable excipients are known and customary in the art and for instance described in Remington; The Science and Practice of Pharmacy, 21^{nd} Edition 2005, University of Sciences in Philadelphia.

A CD40 agonist and optionally a CTL-activating peptide as used in the invention are preferably soluble in physiologically acceptable watery solutions (e.g. PBS) comprising no more than 35 decreasing to 0%; 35, 20, 10, 5 or 0% DMSO. In such a solution, a CD40 agonist is preferably soluble at a concentration of at least 0.5, 1, 2, 4, 6, 8 or 10 mg CD40 agonist per ml. In such a solution, a CTL-activating peptide is preferably soluble at a concentration of at least 0.5, 1, 2, 4, or 8 mg peptide per ml.

Any known immune modulator, may be added to a composition as defined herein. Preferably, the immune modulator is an adjuvant. More preferably, the composition comprises a peptide as earlier defined herein and at least one adjuvant. The adjuvant can be an oil-in-water emulsion such as incomplete Freunds Adjuvants, Montanide ISA51 (Seppic, France), Montanide 720 (Seppic, France) or a TLR ligand, formulated in Montanide or PBS. This type of medicament may be administered as a single administration. Alternatively, the administration of a CD40 agonist and optionally a CTL-activating peptide as earlier herein defined and/or an adjuvant may be repeated if needed and/or distinct CD40 agonists and/or distinct CTL-activating peptides and/or distinct adjuvants may be sequentially administered.

Particularly preferred adjuvants are those that are known to act via the Toll-like receptors (TLR's) (Kawai & S. Akira Signaling to NF-κB by Toll-like receptors Trends in Molecular medicine Vol. 13, p. 460-469, 2007). Adjuvants that are capable of activation of the innate immune system, can be activated particularly well via Toll like receptors (TLR's), including TLR's 1-10 and/or via a RIG-1 (Retinoic acid-inducible gene-1) protein and/or via an endothelin receptor. Compounds capable of activating TLR receptors and modifications and derivatives thereof are well documented in the art. TLR1 may be activated by bacterial lipoproteins and acetylated forms thereof, TLR2 may in addition be activated by Gram positive bacterial glycolipids, LPS, LPA, LTA, fimbriae, outer membrane proteins, heatshock proteins from bacteria or from the host, and Mycobacterial lipoarabinomannans. TLR3 may be activated by dsRNA, in particular of viral origin, or by the chemical compound poly(I:C). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLR5 may be activated with bacterial flagellae or flagellin. TLR6 may be activated by mycobacterial lipoproteins and group B Streptococcus heat labile soluble factor (GBS-F) or Staphylococcus modulins. TLR7 may be activated by imidazoquinolines and derivatives. TLR9 may be activated by unmethylated CpG DNA or chromatin—IgG complexes. In particular TLR3, TLR4, TLR7 and TLR9 play an important role in mediating an innate immune response against viral infections, and compounds capable of activating these receptors are particularly preferred for use in the invention. Particularly preferred adjuvants comprise, but are not limited to, synthetically produced compounds comprising dsRNA, poly(I:C), unmethylated CpG DNA which trigger TLR3 and TLR9 receptors, IC31, a TLR9 agonist, IMSAVAC, a TLR4 agonist. In another preferred embodiment, the adjuvants are physically linked to a peptide as earlied defined herein. Physical linkage of adjuvants and costimulatory compounds or functional groups, to the HLA class I and HLA class II epitope comprising peptides provides an enhanced immune response by simultaneous stimulation of antigen presenting cells, in particular dendritic cells, that internalize, metabolize and display antigen. Another preferred immune modifying compound is a T cell adhesion inhibitor, more preferably an inhibitor of an endothelin receptor such as BQ-788 (Buckanovich R J et al., Ishikawa K, PNAS (1994) 91:4892). BQ-788 is N-cis-2,6-dimethylpiperidinocarbonyl-L-gamma-methylleucyl-D-1-methoxycarbonyltryptophanyl-D-norleucine. However any derivative of BQ-788 or modified BQ-788 compound is also encompassed within the scope of this invention.

Furthermore, the use of APC (co)stimulatory molecules, as set out in WO99/61065 and in WO03/084999, in combination with a CD40 agonist and optionally a CTL-activating peptide present in the medicament used in the invention is preferred. In particular the use of 4-1-BB and/or CD40 ligands, or functional fragments and derivates thereof, as well as synthetic compounds with similar agonistic activity are preferably administered separately or combined with a CD40 agonist and optionally a CTL-activating peptide present in the medicament to a subject to be treated in order to further stimulate the mounting an optimal immune response in the subject.

In a preferred embodiment, the adjuvant comprises an exosome, a dendritic cell, monophosphoryl lipid A and/or CpG nucleic acid.

Therefore in a preferred embodiment, a medicament comprises a CD40 agonist and optionally a CTL-activating peptide as such or present in a composition as earlier defined herein and an adjuvant selected from the group consisting of: oil-in water emulsions (Montanide ISA51, Montanide ISA 720), an adjuvant known to act via a Toll-like receptor, an APC-costimulatory molecule, an exosome, a dendritic cell, monophosphoryl lipid A and a CpG nucleic acid.

In another preferred embodiment, to promote the presentation of a CTL-activating peptide by a professional antigen presenting cell or dendritic cells, a composition or a medicament comprising a peptide further comprises a DC-activating agent.

Ways of administration are known and customary in the art are for instance described in Remington; The Science and Practice of Pharmacy, 21^{st} Edition 2005, University of Sciences in Philadelphia. The administration of a CD40 agonist has been extensively explained herein. The administration of a CTL-activating peptide and/or of any other molecule as used in the invention may be administered the same way as a CD40 agonist (simultaneously or sequentially). Alternatively, a CTL-activating peptide and/or any other molecule may be formulated to be suitable for intravenous or subcutaneous, or intramuscular administration, although other administration routes can be envisaged, such as mucosal administration or intradermal and/or intracutaneous administration, e.g. by injection.

It is furthermore encompassed by the present invention that the administration of at least one CD40 agonist, optionally at least one CTL-activating peptide and/or at least one other molecule or adjuvant as used in the invention may be carried out as a single administration. Alternatively, the administration of at least one CD40 agonist, optionally at least one CTL-activating peptide and/or at least one other molecule or adjuvant as used in the invention may be repeated if needed.

Accordingly, in a further aspect, there is provided a method for treating cancer, a pre-malignant disorder or an infectious disease, wherein an agonist of CD40 is locally administered and targeted to a tumor draining lymph node of a subject. Each feature of this method has already been extensively defined earlier herein. Preferably, in this method, a tumor draining lymph node will be removed after administration of an agonist of CD40. In this context, “after” may mean 7 days or 8, 9, 10, 11, 12, 13, 14, 15, 16, 17, 18 days or longer. A tumor draining lymph node is usually removed as part of a surgical procedure aimed at removing a primary tumor and a lymph node that may contained metastasized tumor cells. This method is attractive since it allows for the tumor specific T cells that are present in a tumor draining lymph node to be activated by CD40 activated DCs. As a consequence of this activation, the tumor-specific T cells will migrate from the tumor draining lymph node to the periphery before this tumor draining lymph node is removed. In this method, cancer is given the same meaning as earlier defined herein. Preferably, in such a method a tumor had been removed by surgery.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a CD40 agonist or a CTL activating peptide as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “approximately” or “about” when used in association with a numerical value (approximately 10, about 10) preferably means that the value may be the given value of 10 more or less 1% of the value.

All patent and literature references cited in the present specification are hereby incorporated by reference in their entirety.

The following examples are offered for illustrative purposes only, and are not intended to limit the scope of the present invention in any way.

### DESCRIPTION OF THE FIGURES

FIG. 1: Systemic anti-CD40 administration causes partially activated tumor specific CTL in the tumor-draining lymph node to proliferate and spread systemically. Tumor-specific CTL stained with tetramer in blood, spleen, tumor-draining and—non-draining lymph nodes with (FIG. 1a) or without systemic agonistic anti-CD40 antibody treatment (FIG. 1b).

FIG. 2: Survival curve of mice with AR6 tumors in right flank, with different administration methods of FGK (agonistic anti-CD40 antibody). The survival of mice injected with of a single dose of 30 μg FGK subcutaneously in tumor draining area is significantly enhanced compared to naïve mice (p=0.002). When FGK a single dose of 30 μg FGK subcutaneously is injected in the non draining area, no beneficial effect is observed (tumor draining vs non draining area, p=0.03). No significant difference in survival was observed between the survival of mice that had received a high dose (3 times 100 μg FGK) intravenously and mice receiving a low dose s.c. in the tumor draining area

FIG. 3: Toxicity of anti-CD40 antibody (FGK) after different administration methods as measured in serum. A: ALAT and ASAT measured in serum from mice at day 1 and 3 after start of anti-CD40 treatment. B & C: cytokine concentration of respectively IL-1b and IL-6 in serum from mice at day 1 and 3 after start of anti-CD40 treatment.

FIG. 4: H&E staining of cryogenic section of different organs, isolated at day 3 after start of treatment.

FIG. 5:

Survival curve of C57BL/6 mice with established subcutaneous palpable syngeneic AR6 (Ad5 E1-induced) tumors either with no antibody (Naive), or with anti-CD40 agonistic antibody (FGK45) as 1) i.v injection at a dose of 100 μg on 3 subsequent days 2) 150 μg in Montanide subcutaneously in the tumor draining area or 3) 150 μg in a non-tumor-draining area on the contralateral flank, 8 mice per group.

FIG. 6:

Detection of Adeno-E1-specific CTL in peripheral venous blood of mice bearing tumors that were either not treated (Naive) or treated with anti-CD40 agonist antibody injected i.v. (FGK IV) or subcutaneously in the tumor-draining area (FGK subcutaneous) as described in FIG. 5, analyzed at day 9 after start of treatment. Blood samples were harvested 9 days after the start of treatment. PBMCs were isolated and stained with CD8 and tetramers. The percentage of tetramer positive CD8⁺ T cells is demonstrated.

FIG. 7:

Toxicity of anti-CD40 antibody (FGK) in serum after different treatment protocols as described in FIG. 5. ALAT (a) and ASAT (b) was measured in serum from mice at day 1, 3, 7 and 21 days after start of anti-CD40 treatment.

FIG. 8:

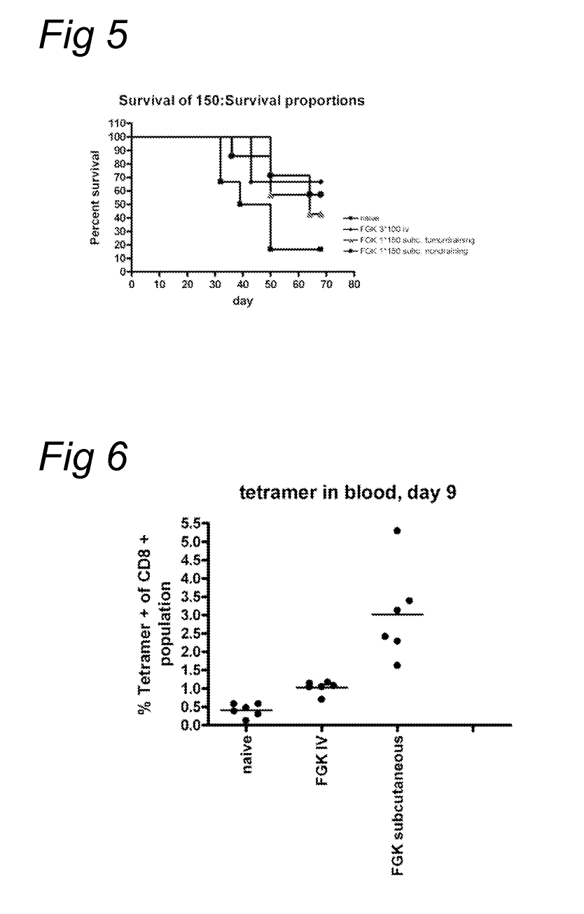

A: Detection of Adeno-E1-specific CTL in peripheral venous blood of mice bearing tumors that were either not treated (Naive) or treated with anti-CD40 agonistic antibody (FGK IV) or in a subcutaneous homolateral tumor-draining area (FGK subcutaneous) as described in FIG. 5. Blood samples were harvested 11 days after the start of treatment. PBMCs were isolated and stained with CD8 and tetramers. The percentage of tetramer positive CD8⁺ T cells is demonstrated. Figure B, C, D and E: examples of flow-cytometry samples of untreated, anti-CD40 high dose intravenous, anti-CD40 low dose slow-release homolateral and anti-CD40 low dose slow-release contralateral, respectively.

FIG. 9:

Mice were treated with different formulations comprising different doses of dextran particles with different water content and anti-CD40 (FGK-45). A: Serum concentration of anti-CD40 were analyzed by ELISA on day 2, 4, 6 and 8. B: Detection of E1A TCR-Tg CTL in peripheral venous blood was analyzed on day 2, 4, 6, 8, 10, 14 and 22. Blood samples were harvested at different times after boost. PBMCs were isolated and stained with CD8 and tetramers. The percentage of tetramer positive CD8⁺ T cells is demonstrated over time.

Doses used were; 30 μg anti-CD40 antibody in Montanide (indicated by a square, 30 montanide), 30 μg anti-CD40 antibody in dextran-particles containing 70% H₂O (indicated by a triangle, 30 70% H₂O), 30 μg anti-CD40 antibody in dextran-particles containing 50% H₂O (indicated by an asterisk and light grey line, 30 50% H₂O), and 30 μg anti-CD40 antibody in a mix of dextran particles containing 70% H₂O, 60% H₂O and 50% H₂O (indicated by an asterisk and black line, 30 mix), 5 μg anti-CD40 antibody in dextran-particles containing 70% H₂O (indicated by a circle, 5 70% H₂O), 5 μg anti-CD40 antibody in dextran particles containing 50% H₂O (indicated by a solid black line, 5 50% H₂O), and 5 μg anti-CD40 antibody in a mix of dextran particles containing 70% H₂O, 60% H₂O and 50% H₂O (indicated by a single stripe and a light grey line, 5 mix).

FIG. 10:

Experiment showing synergy between anti-CD40 antibody and CTLA-4 blocking antibody in Montanide, subcutaneous injection in tumor draining area. Detection of E1A TCR-Tg CTL in peripheral venous blood was analyzed on day 4, 7, and 18. Blood samples were harvested at different times after boost. PBMCs were isolated and stained with CD8 and tetramers. The percentage of tetramer positive CD8⁺ T cells is demonstrated over time.

FIG. 11:

Tumor-specific CTL stained with tetramer in blood over time after a boost with irradiated tumor cells. Tumor-bearing mice were divided into 5 groups. One group of mice was pretreated with local anti-CD40 antibody (FGK-45) in slow-release formulation before tumor and tumor-draining lymph node (T+LN) double resection, the other groups of mice were left untreated before tumor and tumor-draining lymph node resection. Of the four remaining groups, mice in group 2 and 3 had their tumor resected (T), mice in groups 4 and 5 had tumor and tumor-draining lymph node resected (T+LN). Mice of groups 2 and 4 received anti-CD40 antibody (FGK-45) local in slow-release formulation, immediately after surgery, mice in groups 3 and 5 were left untreated. 12 days after surgery, mice received a boost with irradiated tumor cells. CTL response against the tumor cells was analyzed in blood by tetramer staining. Blood samples were harvested at different times after boost. PBMCs were isolated and stained with CD8 and tetramers. The percentage of tetramer positive CD8⁺ T cells is demonstrated over time.

FIG. 12: T-cell response after vaccination with synthetic long peptides in combination with anti-CD40, injected either subcutaneously or intravenously. Mice were vaccinated with synthetic long peptides derived from HPV E7 or CEA in Montanide, in combination with either 30 μg anti-CD40 in the same montanide depot, or 3 times 100 μg anti-CD40 intravenously (on day 0, 1, 2). Mice were boosted with same peptides in Montanide 14 days later, without the addition of anti-CD40 antibody. T-cell response was analyzed in spleen by tetramer-staining. PBMCs were isolated and stained with CD8 and tetramers. The percentage of tetramer positive CD8⁺ T cells is demonstrate. Also intracellular cytokine staining was performed. Both were performed 10 days after the boost vaccination. Per group 5 mice were treated.

A: CD8+ T-cells, positive for HPV E7 tetramer in spleen.

B: CD8+ T-cells, positive for IFN-γ production after HPV E7 peptide stimulation in vitro

C: CD8+ T-cells, double positive for IFN-γ and TNF-α production after HPV E7 peptide stimulation in vitro

D: CD8+ T-cells, positive for IFN-γ production after CEA peptide stimulation in vitro

E: CD8+ T-cells, double positive for IFN-γ and TNF-α production after CEA peptide stimulation in vitro.

FIG. 13: Cytokine concentration in serum after anti-CD40 treatment Mice were untreated (Naive) or injected with either anti-CD40 (30 μg in Montanide) subcutaneously, or intravenously (3 times 100 μg on day 0, 1, 2). On day 1, 3, 7 and 21 after start of treatment, serum samples were collected and analyzed by multiplex assay for cytokine concentrations. Per group 4 mice were treated.

A: IL-2 concentration in serum over time.

B: GM-CSF concentration in serum over time.

### EXAMPLES

In each of the examples herein, an anti-CD40 activating antibody has been used as identified herein.

### Example 1

Low dose anti-CD40 activating therapy in the tumor-draining area is as effective in generating an anti-tumor CTL response as a high dose systemic therapy, with decreased toxicity.

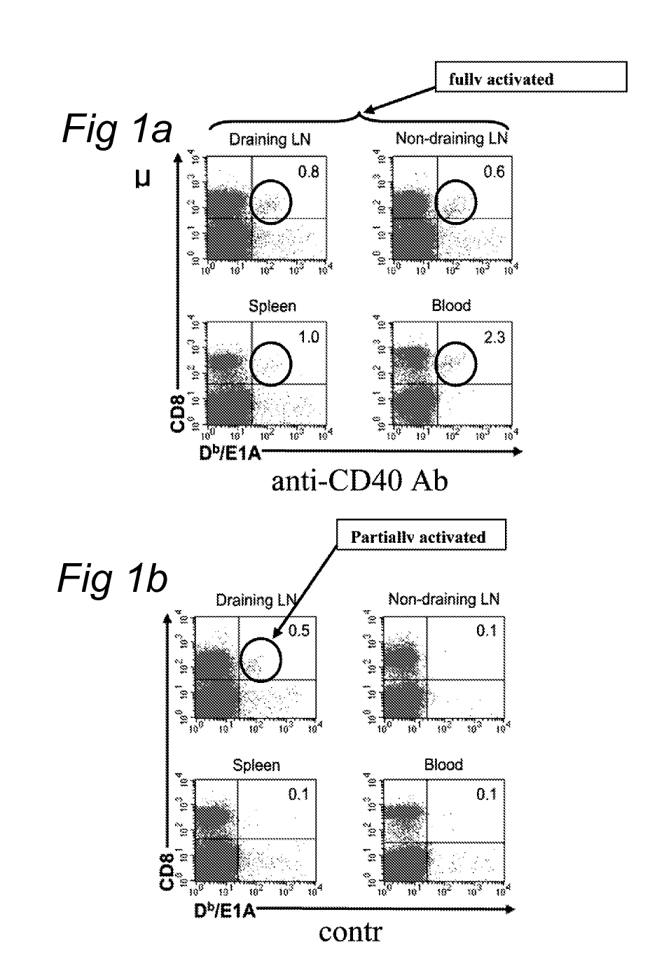

In a mouse-model using adenovirus E1-induced tumor-cells a weak tumor specific CTL response is generated. These CTL persist in the tumor-draining lymph node and are not capable of clearing the tumor. The tumor specific CD8 T-cells are primed by dendritic cells (DC) presenting tumor-antigens in the tumor-draining lymph node. These DC are not activated due to lack of danger signals, such as those delivered to toll-like receptors (TLR, G. J. van Mierlo, et al. J. Immunol. 173, 6753-6759, 2004). By systemically injecting activating anti-CD40 antibodies, the dendritic cells are activated and stimulate the CTL. The tumor specific CTL start proliferating, leave the tumor-draining lymph node and clear the tumor (Van Mierlo et al. (2002) Proc Natl Acad Sci USA 99, 5561; G. J van Mierlo, et al. J. Immunol. 173, 6753-6759, 2004). (FIG. 1).

Systemic injection of anti-CD40 antibody not only activates DC in the tumor-draining area, but DC in the entire body, as well as B-cells, macrophages, and several other cell types. In patients, it causes cytokine-release syndrome and abnormalities in lymphocyte count, platelets, D-dimer (Vonderheide et al. (2007) J Clin Oncol. March 1; 25(7):876-83.) By administration of an anti-CD40 antibody locally in the tumor-draining area instead of systemically, we hypothesized that the dose can be lowered, without loosing effectiveness. We used a slow-release system (Montanide ISA 51, Seppic France) for subcutaneous injection, in order to continuously stimulate CD40 signaling on the tumor antigen presenting DC for several days. The dose we used is ten times lower then the dose used systemically in mice studies. We injected 30 μg of the anti-CD40 antibody in the tumor-draining area, subcutaneously between the tumor and the tumor-draining lymph node. The tumor-model we used is described in materials and methods.

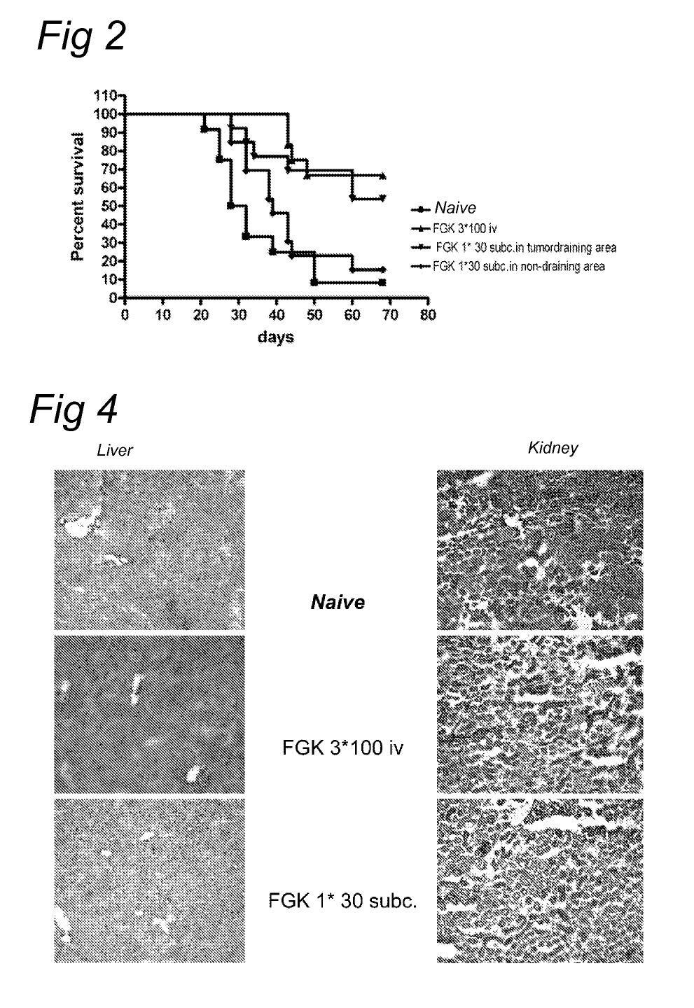

As is shown in FIG. 2, the survival of mice that had received high dose anti-CD40 antibody, injected i.v., was comparable with mice that had received low dose, injected s.c. in tumor-draining area, even though the difference in dose is tenfold. Mice that had received CD40 antibody through either methods of administration showed a significant increase in tumor-clearance compared to naïve mice or mice that received a low dose FGK in the non-draining area (the opposite flank).

This tenfold lower concentration administered in a slow-release emulsion in the tumor-draining area allowed the dose of the therapeutic compound to be high enough at the necessary site, the tumor-draining lymph node, but kept the systemic levels low, which drastically decreases toxicity.

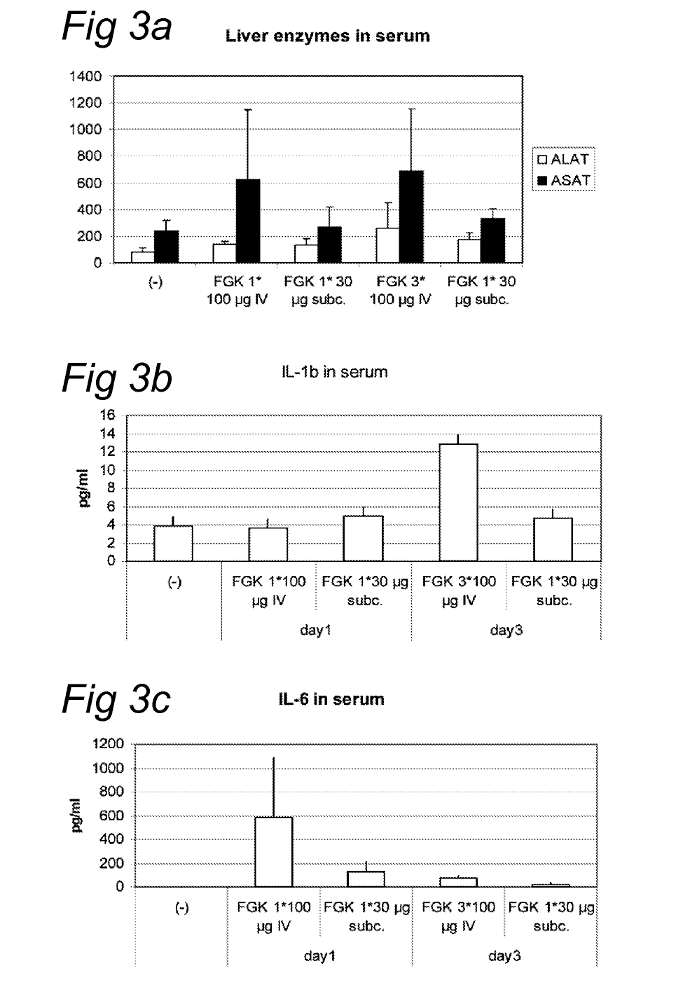

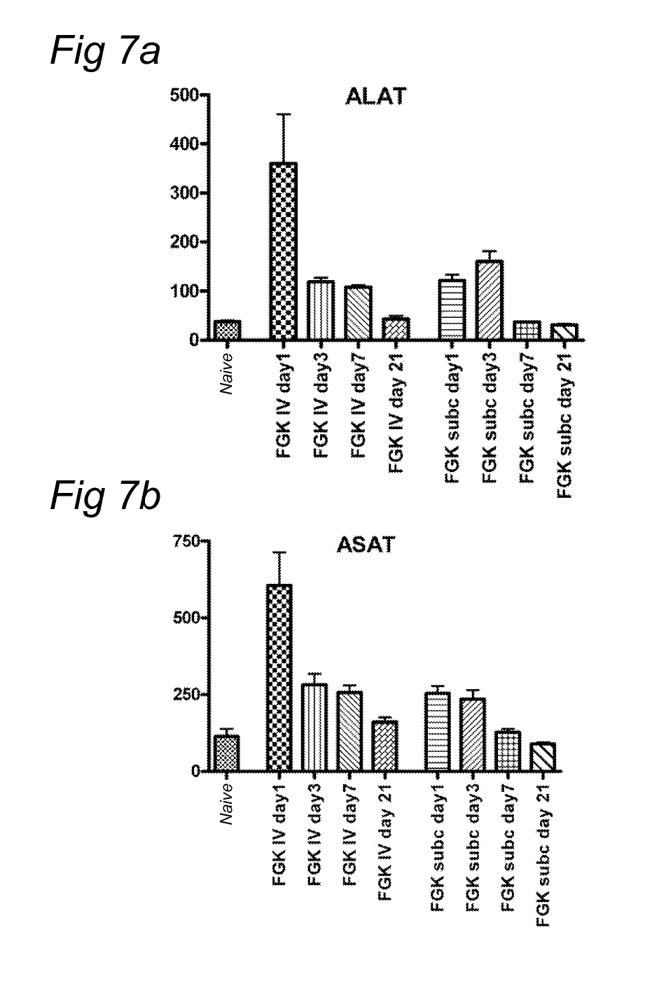

To demonstrate the toxic effects of the different routes of administration and the different doses, systemic toxicity was measured with 3 different assays (FIGS. 3 and 7). ALAT (alanine amino transferase) and ASAT (aspartate amino transferase) levels were determined. These enzymes are present in liver cells. When the liver is damaged, these enzymes are released from dying liver cells in the blood stream and therefore serve as a measure for liver toxicity. IL-1b and IL-6 are cytokines involved in the adverse event called cytokine release syndrome (CRS), elevations of these cytokines in serum are signs of systemic toxicity. FIG. 3a, b, c and FIGS. 7a and b clearly shows that the subcutaneous, local administration leads to lower toxicity than intravenous, systemic administration.

We also studied different organs from mice at day 3 after start of treatment in order to assess levels of toxicity based on histological sections. In FIG. 4 we show that high dose systemic administration of FGK resulted in severe pathology in liver, lung and kidney. Organs show serious edema, tissue damage and loss of physiological organ architecture. Organs of mice that received low dose subcutaneous administration of FGK show only mild signs of toxicity compared to organs from naïve mice. Importantly, the differences in toxicity between high dose systemic administration and low dose subcutaneous administration are significant.

### Materials and Methods:

### Mice

C57BU6 Kh mice were bred and kept in the animal facility of LUMC.

### Tumor Cells

Mouse embryo cells transformed by Ad5EIA plus EJ-ras were cultured in IMDM (Invitrogen Life Technologies, Rockville, Md.) supplemented with 8% (v/v) FCS, 50 μM 2-ME, glutamine, and penicillin.

### Tumor Experiments

CD40-negative E1A-expressing tumor cells (1×10⁷) were injected s.c. in the flank of 7- to 13-wk-old male mice in 200 μl of PBS. Tumor size was measured twice weekly with calipers in three dimensions. Treatment was started 8-18 days after tumor inoculation, when palpable tumors were present. Mice were sacrificed when tumor size exceeded 1 cm³ to avoid unnecessary suffering.

### Treatments

The FGK-45 hybridoma cells producing a stimulatory anti-CD40 Ab were provided by A. Rolink (Basel Institute for Immunology, Basel, Switzerland) Mice received 100 μg of the anti-CD40 mAb given i.v. (days 0, 1, and 2 of treatment) in 200 μl PBS.

Subcutaneous injections were performed in the tumor draining area (under the skin of the flank, between the tumor and the tumor draining lymph node, 200 μl montanide emulsion. Emulsion was made by mixing a 1:1 solution of 0.3 μg/ml FGK in PBS with montanide ISA 51 (Seppic, France) for 30 minutes on a vortex. Final administered dose was 30 μg of FGK.

### Tetramer Staining:

APC-conjugated E1A₂₃₄₋₂₄₃-loaded H-2D^{b} tetramers were used to stain tumor-specific CTL, combined with CD8a staining and analysis was done by flow cytometry.

Hematoxylin and Eosin (H&E) staining:

Cryosections of mouse tissues were stained according to the method described in the IHC world Life Science Network, accessible through Google. The H&E staining protocol was that of Roy Ellis, Division of Pathology, Queen Elizabeth Hospital, Woodville Road, Woodville, South Australia, 5011

### ALAT and ASAT Analyses:

ASAT and ALAT were measured according to the IFCC (International Federation for Clinical Chemistry) recommendations. Reagents are from Roche Diagnostics GmbH (Mannheim, FRG). Cat nr 11876848 for ASAT and nr 11876805 for ALAT. The test principle relies on the decrease of NADH with rising ASAT or ALAT concentration. NADH is measured photometrically. Both enzymes are measured with a fully automated laboratory system on a P800 Modular. (Roche/Hitachi Tokyo, Japan). CV's of these measurements are below 2%.

### Multiplex Array:

Serum samples were collected on day 1, 3, 7 and 21 with heart puncture. Serum was analyzed for the presence of IL-1, IL-6 using the Bio-Plex Pro Mouse Cytokine 23-Plex Panel from Bio-rad using the manufacturer's protocol.

### Example 2

Tumor experiment with higher dose anti-CD40 antibody in the tumor draining area (FIG. 5).

We determined whether it is possible to increase the dose of the anti-CD40 antibody locally, while maintaining the functional effect as measured by tumor clearance and a reduced toxicity. An increased dose, namely 150 μg of FGK-45 was administered s.c. in the tumor draining area or in the flank opposite of the tumor (non-draining area). Survival was compared with mice injected i.v. with 3 times 100 μg of FGK. Interestingly, no difference in survival between the treated mice could be observed when a high dose of FGK was injected. In contrast (see FIG. 2) to the low dose FGK (30 μg in Montanide) where it did matter if the antibody was injected in the tumor draining area or not. This demonstrates that even though the injection is local (s.c.) in a non draining area, a high dose of the antibody ensures that sufficient amounts of antibody reach the periphery through systemic distribution. This is explained by the fact that the dose of anti-CD40 is so high that even though the injection is not near the tumor-draining lymph node, a high enough concentration reaches the tumor-draining lymph node for a tumor-specific response to be activated,

In conclusion, to prevent systemic toxicity, it is not only important to deliver the anti-CD40 antibody locally to the tumor draining lymph node, but also that the dose is such that it ensures uptake by local tissues only, instead of systemic distribution.

### Example 3

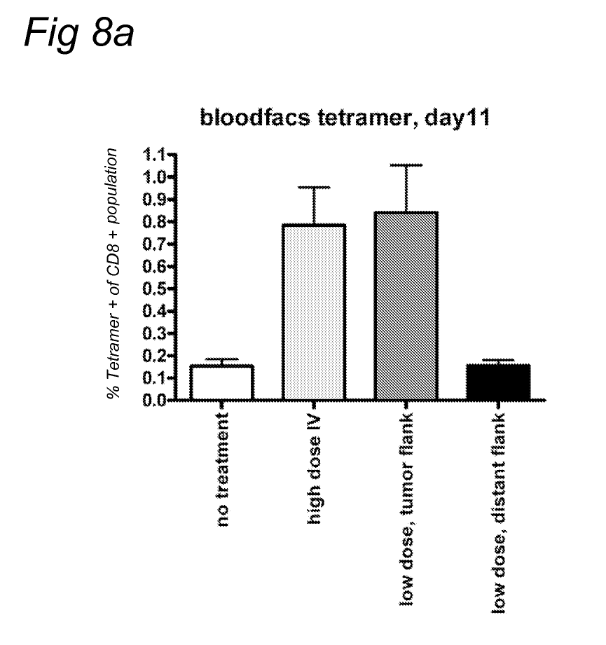

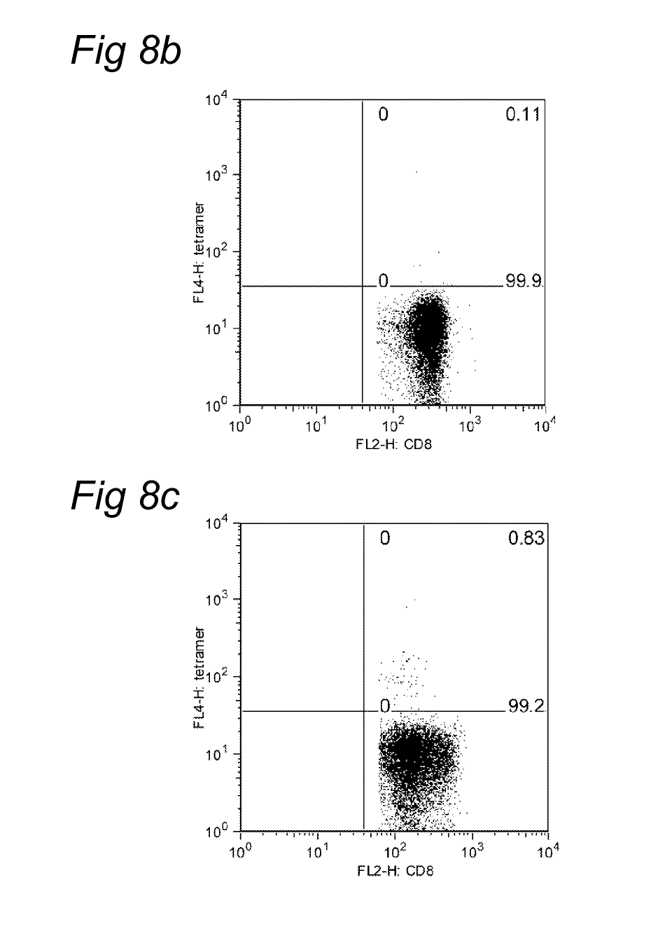

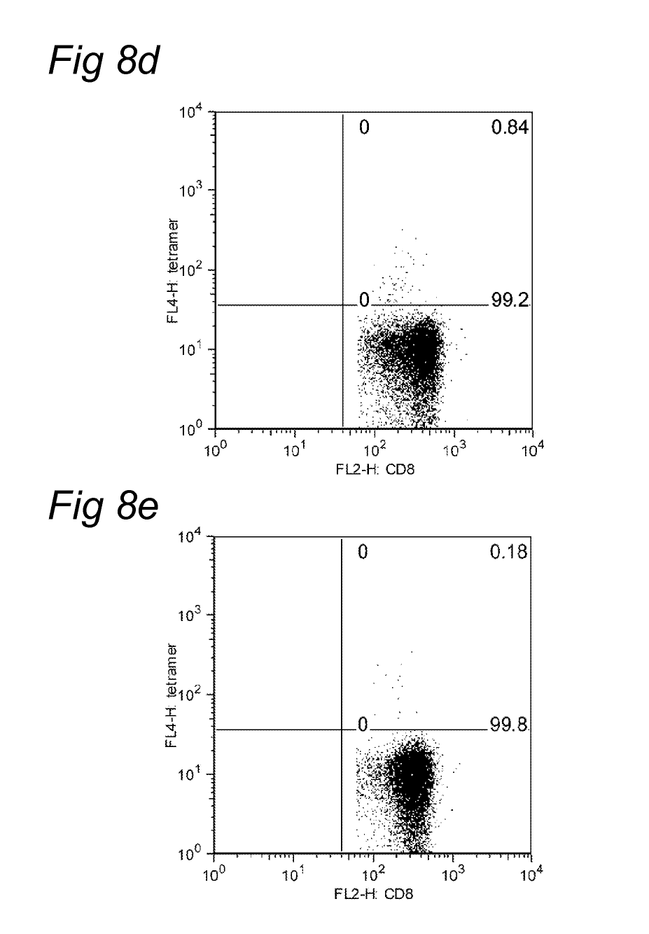

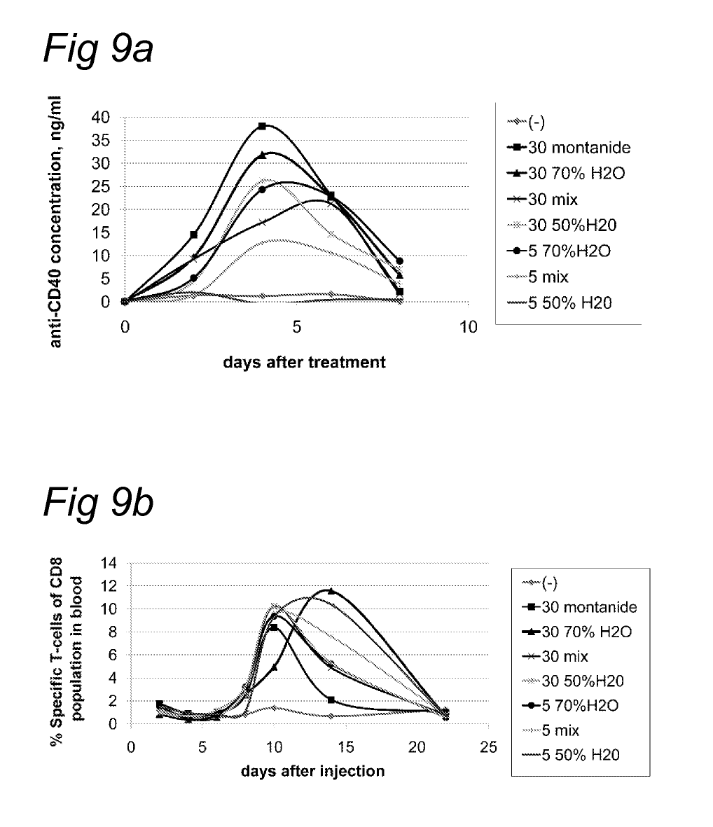

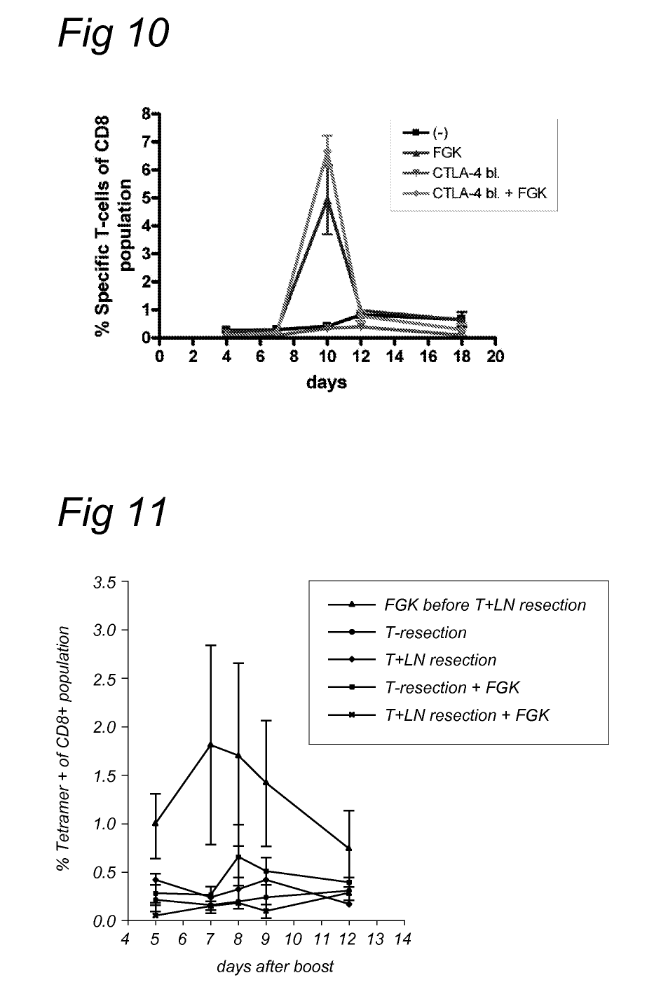

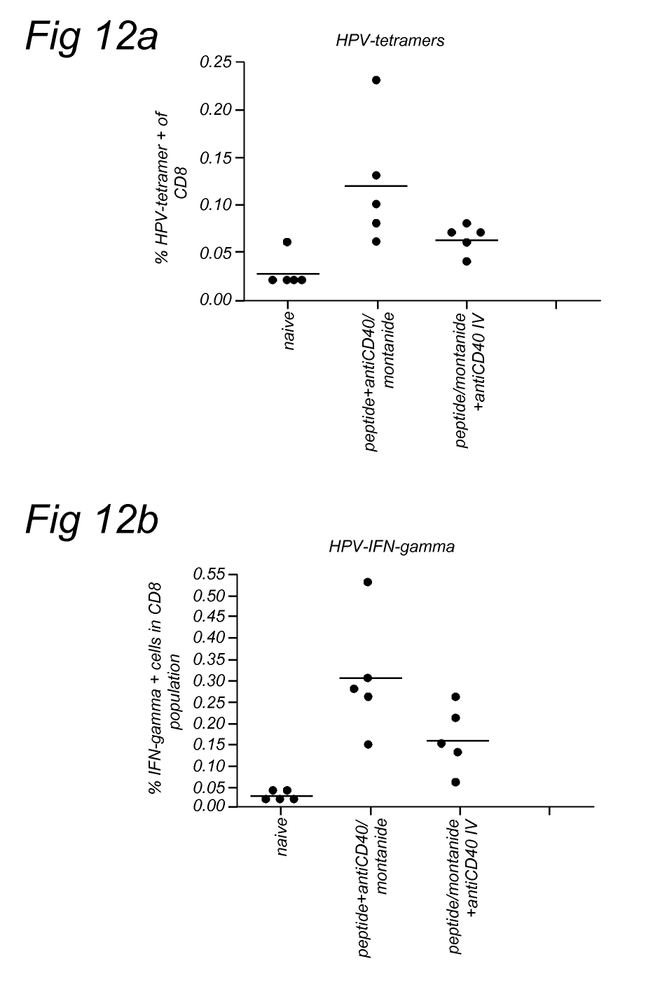

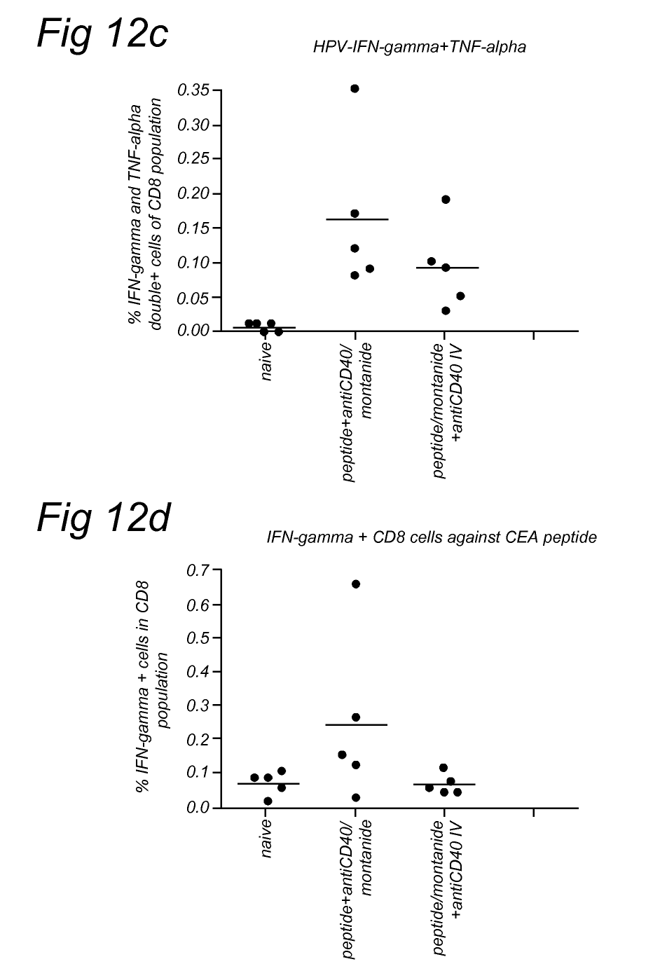

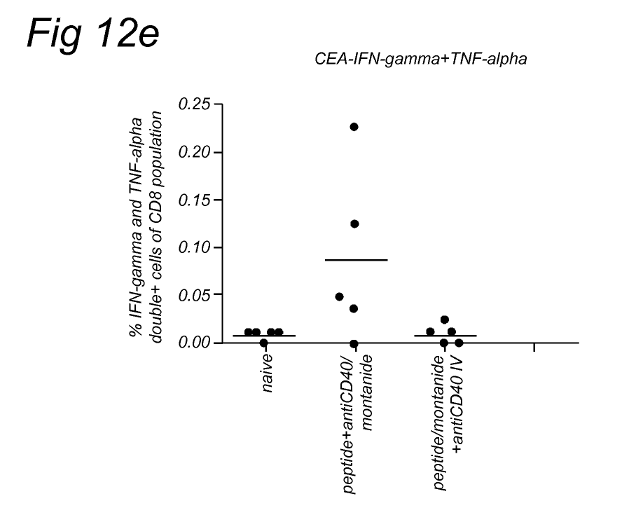

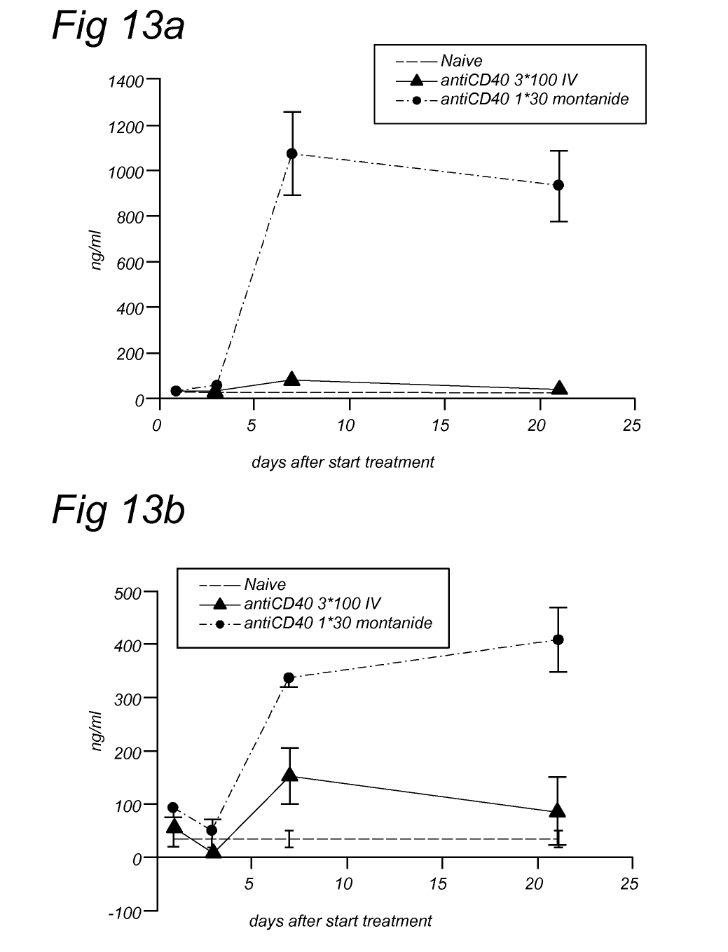

Tetramer staining on blood samples of mice with a subcutaneous tumor (FIGS. 6 and 8).

Blood samples were obtained at day 9 (FIG. 6) or day 11 (FIG. 8) after start of anti-CD40 treatment. The number of tumor-specific CTL were compared between untreated or treated tumor bearing mice. Treated mice received either a high dose anti-CD40 antibody (FGK-45) intravenously (3 times 100 μg) or a low dose subcutaneously (30 g in Montanide) in the tumor draining area or in the contralateral flank (non-draining area). No increase in tetramer positive CD8 T-cells could be detected in the blood of mice treated with anti-CD40 s.c. in the contralateral flank (non-draining area) compared to untreated mice. Importantly, in mice that received systemic anti-CD40 treatment or mice that had received anti-CD40 s.c. in the tumor draining area, clear populations of tetramer positive CD8 T-cells could be demonstrated. This proves that even though the subcutaneous treatment with low dose anti-CD40 is a local treatment (if the treatment is not given in the tumor draining area, it is not effective at this dose), it caused a systemic immune response: induction of tumor specific CTL, detectable in the peripheral blood.

The difference in levels of tetramer positive CD8 T-cells between the subcutaneously treated group and the intravenously treated group could be explained by the enhanced toxicity of the anti-CD40 in the intravenously treated group. It caused severe abnormalities in the numbers of lymphocytes in the blood in the first week after treatment, and it took another week to regain normal levels. (also described in lesser extent in Vonderheide et al).

### Materials and Methods

### Tetramer Staining:

APC-conjugated E1A₂₃₄₋₂₄₃-loaded H-2D^{b} tetramers were used to stain tumor-specific CTL, combined with CD8a staining and analysis was done by flow cytometry.

### Example 4

Dextran-based microparticles as a slow-release system for immunotherapy with anti-CD40 antibody.

Dextran-based microparticles form a slow-release system especially tailored for slow-release of larger proteins, such as antibodies. We used dextran-based microparticles containing an agonistic anti-CD40 antibody (FGK-45) as a slow-release system in experiments with our mouse model, as described in material and methods of example 1. We injected 1×10⁶ E1A TCR Tg CD8 T-cells intravenously into mice bearing E1A expressing tumors, followed by the anti-CD40 containing microparticle-injection. We found that the slowest release formulation, 50% water content, in a low administered dose, 5 μg anti-CD40 antibody (FGK) in dextran particles gave an undetectable concentration of anti-CD40 antibody in the serum (FIG. 9a), but still resulted in measurable activated CTLs in the blood (FIG. 9b).

These data show that the use of dextran-based microparticles as a slow-release system allows for the reduction of the dose of anti-CD40 injected s.c in the tumor draining area without affecting the tumor-specific T cell response. Moreover, the use of reduced concentrations results in reduced concentrations of anti-CD40 antibody in blood, suggesting that the systemic toxicity will also be reduced.

### Material and Methods:

### Addition to Treatments:

Dextran-based particles containing anti-CD40 antibody were prepared as previously described. (O. Franssen, L. Vandervennet, P. Roders, and W. E. Hennink. Chemically degrading dextran hydogels: controlled release of a model protein from.)

Mice were treated with various concentrations of dextran-particles in 200 μl PBS, subcutaneous injections were performed in the tumor draining area (under the skin of the flank, between the tumor and the tumor draining lymph node.

### Addition to Mice:

Mice expressing a TCR specific for the H-2D^{b}-restricted E1A₂₃₄₋₂₄₃ adenoviral epitope (E1A TCR-Tg) were bred and kept in the animal facility of the LUMC.

### Transgenic CTL Analysis:

CD8 T-cells were isolated from spleen and lymph node from E1A TCR Tg-mice with BD Imag lymphocyte enrichtment kit. One million CD8-T-cells were injected intravenously into mice bearing tumors. The kinetics of the CTL response in blood was measured by flowcytometry.

### Antibody Detection in Serum:

Concentration of anti-CD40 antibody in serum was determined by ELISA using anti-rat antibodies.

### Example 5

Synergy between immune activating antibodies in slow-release depot in tumor-draining area.

Administration in a slow-release depot in the tumor-draining area is suitable for other immune-activating antibodies, other than anti-CD40. Combinations of different immune-activating antibodies could lead to an enhanced quality and or quantity of the CTL-response. We injected tumor-bearing mice with 1×10⁶ CD8 T-cells from the tumor-specific TCR-transgenic mouse. Then we combined anti-CD40 antibody FGK-45 with a CTLA-4 blocking antibody (9H10) in a Montanide formulation, and analyzed the kinetics of the peripheral CTL response in blood (FIG. 10). The number of tumor specific CTL in blood was enhanced in mice treated with a combination of anti-CD40 and anti-CTLA-4 antibodies, as compared to treatment with each antibody alone. This suggests that there is synergy between the different antibodies and supports the combination of multiple immune stimulating antibodies in one slow release formulation.

### Example 6

Surgical removal of tumor and tumor-draining lymph node before anti-CD40 local treatment abrogates the anti-tumor CTL response.

In the clinic, tumors and tumor-draining lymph node (LN) are generally resected surgically as part of the treatment. We hypothesized that both the tumor and the tumor-draining LN are necessary for a successful local immune-activating antibody treatment, and therefore the treatment should be started before tumor and tumor-draining LN resection.

To demonstrate this, we inoculated 5 groups of mice with tumor-cells. When tumors were palpable, we treated group 1 with local anti-CD40 in montanide. 12 days later, all mice underwent surgery. Tumor and tumor-draining LN were resected in mice in group 1, 2 and 3, only the tumor was resected in mice in group 4 and 5. Mice from group 2 and 4 received anti-CD40 immediately after the resection. Mice from group 3 and 5 didn't receive any anti-CD40. 12 days after surgery, all mice received a boost vaccination of irradiated tumor-cells in the opposite flank. Blood samples were taken regularly to analyze the anti-tumor CTL response by tetramer-staining.

As is shown in the FIG. 11, there is a good CTL response in the mice that received anti-CD40 before tumor and tumor-draining LN resection, but not in any of the other groups. Therefore, it may be important to perform this type of treatment when tumor draining lymph nodes are still present.

### Material and Methods:

Mice were anesthetized with ketamine en xylazine (1:1:2 in PBS, 100 microliter intraperitoneal). Tumor and tumor-draining lymph node were isolated, and wounds were closed with woundclips. 5 days later clips were removed. Mice received a boost vaccination,

### Example 7

Previously, we have published that the addition of anti-CD40 activating antibodies has a positive effect on priming of CTL against peptides in vaccination setting (Diehl et al. Nat Med. 1999 July; 5(7):774-9). Therefore, we tested whether the combination of anti-CD40 and long HPV-derived peptide (Bijker et al, JJ Immunol. 2007 Oct. 15; 179(8):5033-40) containing a CTL epitope in one single slow release formulation, given locally had a similar effect on T cell priming as the previously used systemic administration of anti-CD40 in combination with a CTL peptide in a separate, slow release formulation. Mice were injected s.c. with 30 μg anti-CD40 and HPV long peptide in Montanide or received i.v. injection of 3 times 100 μg anti-CD40 and simultaneously a s.c. injection of HPV long peptide in Montanide. T cell response was measured 10 days after a booster peptide vaccination in the spleen of the treated mice. We show that using a low dose, local slow-release formulation of anti-CD40 antibody (FGK-45)is more effective as adjuvant in peptide vaccination as high dose intravenous injection. Both for the long synthetic HPV peptide and the long synthetic CEA peptide, the response was enhanced both in quantity and quality of CD8+ T-cells in mice treated with low dose, local slow release formulation of anti-CD40 antibody (FGK-45) compared to high dose, intravenous injections. (FIG. 12a, b, c).

To determine whether this observation can be expanded towards other long peptides containing a CTL epitope, the same experiment was performed using a long peptide derived from CEA, another tumor associated protein that is overexpressed in some epithelial cancers. The same observations were demonstrated using this peptide (FIG. 12 d and e).

In conclusion, both for the long synthetic HPV peptide and the long synthetic CEA peptide, the response was enhanced both in quantity and quality of CD8+ T-cells in mice treated with low dose, local slow release formulation of anti-CD40 antibody as compared to high dose, intravenous injections.

### Example 8

Cytokines can contribute to a better immune response against pathogens or tumors, or improve the survival of specific T-cells. In therapeutic setting, such as vaccination and cancer treatment, cytokines are sometimes given as adjuvant to patients. Examples of such immune boosting cytokines are IL-2 and GM-CSF. We have determined the concentration of these cytokines in the serum of mice after treatment with anti-CD40 activating antibody (FGK-45), either in low dose, slow release formulation injected subcutaneously in the tumor draining area, or high dose intravenous injections (as described earlier in examples 1-3). We show that for both IL-2 (FIG. 13a) and GM-CSF (FIG. 13b), the levels are strongly elevated in mice after treatment with low dose, slow release formulated anti-CD40 antibody, even after a prolonged time, compared to high dose intravenous injections. This again indicates that the local delivery of anti-CD40 at a low dose in the tumor draining area is superior to systemic administration of anti-CD40 with respect to the induction of beneficial cytokines after treatment.

### Material and Methods:

### Peptide Vaccination:

40 nmol of each peptide (HPV E7: GQAEPDRAHYNIVTFCCKCDSTLRLCVQSTHVDIR or CEA: VTRNDARAYVCGIONSVSANRSDPV, (with the CTL epitope indicated in bold) was injected in a 1:1 emulsion of PBS and Montanide, 200 μl subcutaneously. One group of mice also received 30 μg of anti-CD40 activating antibody in the same Montanide formulation as the peptide. The second group of mice received a peptide in Montanide depot, which was injected s.c. and a simultaneous injection of 100 μg anti-CD40 activating antibody intravenously on day 0. This was followed by additional injections of 100 μg anti-CD40 i.v. on day 1 and 2. 14 days later mice received a boost vaccination consisting of 40 nmol of each peptide, injected in a 1:1 emulsion of PBS and Montanide, 200 μl subcutaneously, in the contralateral flank. No anti-CD40 antibody was given at this time.

### Intracellular Cytokine Staining:

Spleen cells were isolated and stimulated overnight with 5 μg/ml of the synthetic long peptide. The Becton Dickinson Cytofix/Cytoperm kit was used for the staining. Samples were analyzed by flow cytometry. Flow cytometry antibodies used were: anti CD3, anti-CD4, anti CD8, anti-IFN-γ and anti-TNF-α, all from Becton Dickinson.

### Multiplex Array:

Serum samples were collected on day 1, 3, 7 and 21 with heart puncture. Serum was analyzed for the presence of IL-2 and GM-CSF using the Bio-Plex Pro Mouse Cytokine 23-Plex Panel from Bio-rad using the manufacturer's protocol.

1-15. (canceled)

16. A method for treating a tumor or cancer, a pre-malignant disorder or an infection in a subject, comprising targeting an agonist of CD40 to a lymph node (LN) draining a site of said tumor or cancer, pre-malignant disorder or infection in said subject by locally administering the agonist to said site,
wherein, when the subject is one with a tumor or cancer, the agonist is not administered intratumorally.

17. The method according to claim 16 for treating a tumor or cancer in the subject, wherein the draining LN is a tumor-draining LN.

18. The method according to claim 16, wherein the agonist is administered subcutaneously.

19. The method according to claim 16, wherein the agonist is administered intracutaneously.

20. The method according to claim 16, wherein the agonist is targeted to said draining LN via injection into a lymphatic vessel.

21. The method according claim 17 wherein the agonist is targeted to said tumor-draining LN via injection into a lymphatic vessel.

22. The method according to claim 16, wherein the agonist is an anti-CD40 antibody or a CD40-binding fragment thereof, a peptide, an oligonucleotide or another small organic molecule.

23. The method according to claim 17, wherein the agonist is an anti-CD40 antibody or a CD40-binding fragment thereof, a peptide, an oligonucleotide or another small organic molecule.

24. The method according to claim 22, wherein the agonist is an anti-CD40 antibody.

25. The method according to claim 24 wherein the anti-CD40 antibody is a human, humanized, chimeric or deimmunized antibody.

26. The method according to claim 17, wherein an effective local dose of the agonist targeted to the tumor-draining LN is 25% to 50% of an effective systemic dose of the agonist for treating said tumor or cancer.

27. The method according to claim 16, wherein the agonist is administered as a single dose.

28. The method according to claim 17, wherein the dose of the agonist does not exceed 90 μg.

29. The method according to claim 16, wherein the agonist is formulated as a slow release formulation.

30. The method according to claim 17, wherein the agonist is formulated as a slow release formulation.

31. The method according to claim 28, wherein the agonist is formulated as a slow release formulation.

32. The method according to claim 16 that further comprises administering to the subject a CTL activating peptide and/or a second stimulating compound.

33. The method according to claim 17 that further comprises administering to the subject a CTL activating peptide and/or a second stimulating compound.

34. The method according to claim 30, wherein the second stimulating compound is a CTLA4-blocking antibody.

35. The method according to claim 17, wherein the targeted tumor-draining LN is removed after administration of said agonist.


SEQUENCE LISTING (9 sequences)

SEQ ID NO: 1 - protein, 365 aa, Human Papilloma Virus-16 E2 protein
METLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKCLRYRFKKHCTLYTAVSSTWHWTGHNVKHKSAIVTLTYDSEWQRDQFLSQVKIPKTITVSTGFMSI

SEQ ID NO: 2 - protein, 158 aa, Human Papilloma Virus-16 E6 protein
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 3 - protein, 98 aa, Human Papilloma Virus-16 E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 4 - protein, 365 aa, Human Papilloma Virus-18 E2 protein
MQTPKETLSERLSCVQDKIIDHYENDSKDIDSQIQYWQLIRWENAIFFAAREHGIQTLNHQVVPAYNISKSKAHKAIELQMALQGLAQSRYKTEDWTLQDTCEELWNTEPTHCFKKGGQTVQVYFDGNKDNCMTYVAWDSVYYMTDAGTWDKTATCVSHRGLYYVKEGYNTFYIEFKSECEKYGNTGTWEVHFGNNVIDCNDSMCSTSDDTVSATQLVKQLQHTPSPYSSTVSVGTAKTYGQTSAATRPGHCGLAEKQHCGPVNPLLGAATPTGNNKRRKLCSGNTTPIIHLKGDRNSLKCLRYRLRKHSDHYRDISSTWHWTGAGNEKTGILTVTYHSETQRTKFLNTVAIPDSVQILVGYMTM

SEQ ID NO: 5 - protein, 158 aa, Human Papilloma Virus-18 E6 protein
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 6 - protein, 105 aa, Human Papilloma Virus-18 E7 protein
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 7 - protein, 393 aa, homo sapiens [sequence omitted]

SEQ ID NO: 8 - protein, 35 aa, artificial
  Fragment of Human Papillomavirus-16 E7 protein
GQAEPDRAHYNIVTFCCKCDSTLRLCVQSTHVDIR

SEQ ID NO: 9 - protein, 25 aa, artificial
  Fragment of Carcinoembryonic antigen protein
VTRNDARAYVCGIQNSVSANRSDPV